SEQUENCE LISTING (3 sequences)

SEQ ID NO: 1 - DNA, 4412 bp, fetal brain
  CDS (45)..(4214): cDNA library from fetal brain
gatctctctccggtcgcgcacgccgaggccagtagggagagaagatggtggttctccgcagcagcttggagctgcacaaccactccgcggcctcggccacgggctccttggacctgtccagtgacttcctcagtctggagcacatcggccggaggcggctccgctcggccggcgcggcgcagaagaaacccgcggcgaccacagccaaagcgggcgatgggtcatcagttaaggaagttgaaacctaccaccggacacgtgctttaagatctttgagaaaagatgcacagaattcttcagattctagttttgagaagaatgtggaaataacggagcaacttgctaatggcaggcattttacaaggcagttggccagacagcaggctgataaaaaaaaagaagagcacagagaagacaaagtgattccagttactcggtcattgagggctagaaacatcgttcaaagtacagaacacttacatgaagataatggtgatgttgaagtgcgtcgaagttgtaggattagaagtcgttatagtggtgtaaaccagtccatgctgtttgacaaacttataactaacactgctgaagctgtacttcaaaaaatggatgacatgaagaagatgcgtagacagcgaatgagagaacttgaagacttgggagtgtttaatgaaacagaagaaagcaatcttaatatgtacacaagaggaaaacagaaagatattcaaagaactgatgaagaaacaactgataatcaagaaggcagtgtggagtcatctgaagagggtgaagaccaagaacatgaagatgatggtgaagatgaagatgatgaagatgatgatgatgatgacgatgatgatgatgatgatgatgatgaagatgatgaagatgaagaagatggagaagaagagaatcagaagcgatattatcttagacagagaaaagctactgtttactatcaggctccattggaaaaacctcgtcaccagagaaagcccaacatattttatagtggcccagcttctcctgcaagaccaagataccgattatcttccgcaggaccaagaagtccttactgtaaacgaatgaacaggcgaaggcatgcaatccacagtagtgactcgacttcatcttcctcctctgaagatgaacagcactttgagaggcggaggaaaaggagtcgtaatagggctatcaataggtgcctcccactaaattttcggaaagatgaattaaaaggcatttataaagatcgaatgaaaattggagcaagccttgccgatgttgatccaatgcaactagattcttcagtacgatttgatagtgttggtggcctgtctaatcatatagcagctctaaaagagatggtggtgtttccattactttatccagaagtctttgaaaaatttaaaattcaacccccaagaggttgtttgttttatgggccacctggaactggaaagactctggttgccagagcacttgccaatgagtgcagtcaaggggataaaagagtagcatttttcatgaggaaaggtgctgattgtctaagtaaatgggtaggagaatctgaaagacagctacgattgctgtttgatcaggcctatcagatgcgcccatcaattattttttttgacgaaattgatggtctggctccagtacggtcaagcaggcaagatcagattcacagttctattgtttccaccctgctagctcttatggatggattggacagcagaggggaaattgtggtcattggtgctacgaacaggctagattctatagatcctgctttacgaaggcctggtcgctttgatagagaattcctctttagcctgcctgataaagaggctcgaaaagagattctaaagattcacaccagggattggaatcccaaaccactggacacatttttagaagagctagcagaaaactgtgttggatactgtggagcagatattaaatcaatatgtgctgaagctgctttatgtgctttacgacgacgctacccacagatctataccactagtgagaaactgcagttggatctctcttcaattaatatctcagctaaggatttcgaggtagctatgcaaaagatgataccagcctcccaaagagctgtgacatcacctgggcaggcactgtccaccgttgtgaaaccactcctgcaaaacactgttgacaagattttagaagccctgcagagagtatttccacatgcagaattcagaacaaataaaacattagactcagatatttcttgtcctctgctagaaagtgacttggcttacagtgatgatgatgttccatcagtttatgaaaatggactttctcagaaatcttctcataaggcaaaagacaattttaattttcttcatttgaatagaaatgcttgttaccaacctatgtcttttcgaccaagaatattgatagtaggagaaccaggatttgggcaaggttctcacttggcaccagctgtcattcatgctttggaaaagtttactgtatatacattagacattcctgttctttttggagttagtactacatcccctgaagaaacatgtgcccaggtgattcgtgaagctaagagaacagcaccaagtatagtgtatgttcctcatatccacgtgtggtgggaaatagttggaccgacacttaaagccacatttaccacattattacagaatattccttcatttgctccagttttactacttgcaacttctgacaaaccccattccgctttgccagaagaggtgcaagaattgtttatccgtgattatggagagatttttaatgtccagttaccggataaagaagaacggacaaaattttttgaagatttaattctaaaacaagctgctaagcctcctatatcaaaaaagaaagcagttttgcaggctttggaggtactcccagtagcaccaccacctgagccaagatcactgacagcagaagaagtgaaacgactagaagaacaagaagaagatacatttagagaactgaggattttcttaagaaatgttacacataggcttgctattgacaagcgattccgagtgtttactaagcctgttgaccctgatgaggttcctgattatgtcactgtaataaagcaaccaatggacctttcatctgtaatcagtaaaattgatctacacaagtatctgactgtgaaagactatttgagagatattgatctaatctgtagtaatgccttagaatacaatccagatagagatcctggagatcgtcttattaggcatagagcctgtgctttaagagatactgcctatgccataattaaagaagaacttgatgaagactttgagcagctctgtgaagaaattcaggaatctagaaagaaaagaggttgtagctcctccaaatatgccccgtcttactaccatgtgatgccaaagcaaaattccactcttgttggtgataaaagatcagacccagagcagaatgaaaagctaaagacaccgagtactcctgtggcttgcagcactcctgctcagttgaagaggaaaattcgcaaaaagtcaaactggtacttaggcaccataaaaaagcgaaggaagatttcacaggcaaaggatgatagccagaatgccatagatcacaaaattgagagtgatacagaggaaactcaagacacaagtgtagatcataatgagaccggaaacacaggagagtcttcggtggaagaaaatgaaaaacagcaaaatgcctctgaaagcaaactggaattgagaaataattcaaatacttgtaatatagagaatgagcttgaagactctaggaagactacagcatgtacagaattgagagacaagattgcttgtaatggagatgcttctagctctcagataatacatatttctgatgaaaatgaaggaaaagaaatgtgtgttctgcgaatgactcgagctagacgttcccaggtagaacagcagcagctcatcactgttgaaaaggctttggcaattctttctcagcctacaccctcacttgttgtggatcatgagcgattaaaaaatcttttgaagactgttgttaaaaaaagtcaaaactacaacatatttcagttggaaaatttgtatgcagtaatcagccaatgtatttatcggcatcgcaaggaccatgataaaacatcacttattcagaaaatggagcaagaggtagaaaacttcagttgttccagatgatgatgtcatggtatcgagtattctttatattcagttcctatttaagtcatttttgtcatgtccgcctaattgatgtagtatgaaaccctgcatctttaaggaaaagattaaaatagtaaaataaaagtatttaaactttcctgatatttatgtacatattaagataaatgtcatgtgtaagataactgataaata

SEQ ID NO: 2 - protein, 1390 aa, fetal brain
MVVLRSSLELHNHSAASATGSLDLSSDFLSLEHIGRRRLRSAGAAQKKPAATTAKAGDGSSVKEVETYHRTRALRSLRKDAQNSSDSSFEKNVEITEQLANGRHFTRQLARQQADKKKEEHREDKVIPVTRSLRARNIVQSTEHLHEDNGDVEVRRSCRIRSRYSGVNQSMLFDKLITNTAEAVLQKMDDMKKMRRQRMRELEDLGVFNETEESNLNMYTRGKQKDIQRTDEETTDNQEGSVESSEEGEDQEHEDDGEDEDDEDDDDDDDDDDDDDDEDDEDEEDGEEENQKRYYLRQRKATVYYQAPLEKPRHQRKPNIFYSGPASPARPRYRLSSAGPRSPYCKRMNRRRHAIHSSDSTSSSSSEDEQHFERRRKRSRNRAINRCLPLNFRKDELKGIYKDRMKIGASLADVDPMQLDSSVRFDSVGGLSNHIAALKEMVVFPLLYPEVFEKFKIQPPRGCLFYGPPGTGKTLVARALANECSQGDKRVAFFMRKGADCLSKWVGESERQLRLLFDQAYQMRPSIIFFDEIDGLAPVRSSRQDQIHSSIVSTLLALMDGLDSRGEIVVIGATNRLDSIDPALRRPGRFDREFLFSLPDKEARKEILKIHTRDWNPKPLDTFLEELAENCVGYCGADIKSICAEAALCALRRRYPQIYTTSEKLQLDLSSINISAKDFEVAMQKMIPASQRAVTSPGQALSTVVKPLLQNTVDKILEALQRVFPHAEFRTNKTLDSDISCPLLESDLAYSDDDVPSVYENGLSQKSSHKAKDNFNFLHLNRNACYQPMSFRPRILIVGEPGFGQGSHLAPAVIHALEKFTVYTLDIPVLFGVSTTSPEETCAQVIREAKRTAPSIVYVPHIHVWWEIVGPTLKATFTTLLQNIPSFAPVLLLATSDKPHSALPEEVQELFIRDYGEIFNVQLPDKEERTKFFEDLILKQAAKPPISKKKAVLQALEVLPVAPPPEPRSLTAEEVKRLEEQEEDTFRELRIFLRNVTHRLAIDKRFRVFTKPVDPDEVPDYVTVIKQPMDLSSVISKIDLHKYLTVKDYLRDIDLICSNALEYNPDRDPGDRLIRHRACALRDTAYAIIKEELDEDFEQLCEEIQESRKKRGCSSSKYAPSYYHVMPKQNSTLVGDKRSDPEQNEKLKTPSTPVACSTPAQLKRKIRKKSNWYLGTIKKRRKISQAKDDSQNAIDHKIESDTEETQDTSVDHNETGNTGESSVEENEKQQNASESKLELRNNSNTCNIENELEDSRKTTACTELRDKIACNGDASSSQIIHISDENEGKEMCVLRMTRARRSQVEQQQLITVEKALAILSQPTPSLVVDHERLKNLLKTVVKKSQNYNIFQLENLYAVISQCIYRHRKDHDKTSLIQKMEQEVENFSCSR

SEQ ID NO: 3 - protein, 578 aa, fetalbrain
  ARAP11 fragment from amino acid residues 813- 1390
VIHALEKFTVYTLDIPVLFGVSTTSPEETCAQVIREAKRTAPSIVYVPHIHVWWEIVGPTLKATFTTLLQNIPSFAPVLLLATSDKPHSALPEEVQELFIRDYGEIFNVQLPDKEERTKFFEDLILKQAAKPPISKKKAVLQALEVLPVAPPPEPRSLTAEEVKRLEEQEEDTFRELRIFLRNVTHRLAIDKRFRVFTKPVDPDEVPDYVTVIKQPMDLSSVISKIDLHKYLTVKDYLRDIDLICSNALEYNPDRDPGDRLIRHRACALRDTAYAIIKEELDEDFEQLCEEIQESRKKRGCSSSKYAPSYYHVMPKQNSTLVGDKRSDPEQNEKLKTPSTPVACSTPAQLKRKIRKKSNWYLGTIKKRRKISQAKDDSQNAIDHKIESDTEETQDTSVDHNETGNTGESSVEENEKQQNASESKLELRNNSNTCNIENELEDSRKTTACTELRDKIACNGDASSSQIIHISDENEGKEMCVLRMTRARRSQVEQQQLITVEKALAILSQPTPSLVVDHERLKNLLKTVVKKSQNYNIFQLENLYAVISQCIYRHRKDHDKTSLIQKMEQEVENFSCSR

(19) United States
(12) Patent Application Publication
(10) Pub. No.: US 2003/0124508 A1
(43) Pub. Date: Jul. 3, 2003

(54) Method for testing the hormonal effect of substances

(76) Inventors: Maik Obendorf, Weimar (DE); Siegmund Wolf, Jena (DE); Jens Schroder, Jena (DE)

Correspondence Address: Eugene E. Renz, Jr., Eugene E. Renz, Jr., P.C., 205 North Monroe Street, Post Office Box 2056, Media, PA, 19063-9056, US

(21) Appl. No.: 10/137,418

(22) Filed: May 3, 2002

(30) Foreign Application Priority Data
May 4, 2001 (DE) 101 21 710.2
Dec. 13, 2001 (DE) 101 61 325.3

(51) Int. Cl. C12Q 1/00; C12Q 1/68

(52) U.S. Cl. 435/4; 435/6

(57) ABSTRACT

A method for testing the hormonal effect, especially the androgenic or antiandrogenic effect, of substances is described, in which (a) cells which are transfected with two vectors, one of these vectors containing DNA which codes for a nuclear receptor protein or a fragment thereof, whereas the other vector contains DNA which codes for a co-modulator or a fragment thereof, are exposed to the substance; and (b) the transcription activity which the nuclear receptor or its fragment induces in the presence of the co-modulator or its fragment and/or the effect of the substance on the interaction between the receptor or its fragment and the co-modulator or its fragment is measured by the protein-protein interaction or the protein-protein-DNA interaction. In addition, a method for determining defects in the co-modulation mechanism and means suitable for performing this method are provided.

### BACKGROUND OF THE INVENTION

It is well known that a class of compounds known as androgens are the hormonal signals responsible for maleness in mammals in general and human beings in particular. As with most hormonal signals, androgens interact with their targets by binding to a receptor, known as the androgen receptor. Recognition of androgens by the androgen receptor starts a series of transcriptional events giving rise to male-associated processes in certain organs and tissues. The binding of androgens to the androgen receptor is also an important process in many androgen related diseases and conditions, such as baldness and acne, as well as important clinical diseases such as prostate cancer. The androgen receptor belongs to the steroid receptor super family that plays a significant role in male sexual differentiation and prostate cell proliferation. Abnormal expressions or mutations of the androgen receptor in prostate cells may play an important role in the progression of prostate cancer.

When bound to androgens and androgen responsive elements, the androgen receptor can up-regulate or down-regulate the expression of androgen target genes through a complicated process that may involve multiple adaptors or co-activators. An important problem in the field of steroid hormone regulation is the question or how specific androgen-activated transcription can be achieved in vivo when several different receptors recognize the same DNA sequence. For example, the androgen receptor (AR), the glucocorticoid receptor (GR) and the progesterone receptor (PR) all recognize the same sequence but activate different transcription activities. It has been speculated by some that accessory factors may selectively interact with the androgen receptor to determine the specificity of the androgen receptor target gene activation.

One of the important uses for the androgen receptor is for testing the androgenic or anti-androgenic effects of specific candidate human pharmaceutical molecules. The androgenic effect of pharmaceuticals is usually an attribute of potential candidate therapeutic medicines that must be evaluated during the process of evaluating a molecule for human therapeutic value. Accordingly, the androgen receptor is used in screens to determine the frequency and specificity by which specific molecules bind to such receptors.

### DESCRIPTION OF THE INVENTION

The present invention relates generally to a method for testing the hormonal effect of substances. More specifically, the invention is directed to a method for determining defects in the co-modulation mechanism of nuclear receptors, and to means suitable for implementing these methods, especially the co-activator ARAP11 for the human androgen receptor and for other nuclear receptors as well as the DNA coding for these means.

When substances are evaluated for their biological activity with respect to their possible pharmaceutical applications, it is general practice to test these substances for any possible hormonal effects they may have, especially for any androgenic or anti-androgenic activity. When pharmacologically active substances are administered, knowledge concerning the hormonal effects, especially the androgenic and anti-androgenic effects, of these substances are often important, because they can cause adverse side effects in the patient. To test the hormonal action of substances, it is possible in particular to use methods which measure the ability of the substances to bind to hormone receptors and to activate their transcriptional activity.

Knowledge of the hormonal effects of substances is of interest not only in the case of potential drugs but also in the case of non-pharmaceutical substances, because it is assumed that many substances present in the environment can show androgenic or anti-androgenic effects or estrogenic or anti-estrogenic effects in certain portions of the population. It is therefore possible for undesirable, harmful effects to be produced.

There is therefore a very considerable need for a method and a means suitable for implementing the method by means of which information concerning the hormonal effect of substances can be obtained in a reliable, sensitive, simple, low-cost, and rapid manner. The methods known so far do not meet these requirements.

The present invention is therefore based on the task of providing a method and means suitable for implementing the method by means of which information concerning the hormonal effect of substances to be tested can be obtained in a reliable, sensitive, simple, low-cost, and rapid manner.

The objective of the invention is achieved surprinsingly by a method for testing the hormonal effect, especially of the androgenic or anti-androgenic effect, of substances in which:

(a) cells which are transfected with two vectors, one of which contains DNA coding for a nuclear receptor protein or a fragment thereof, especially a human nuclear receptor or a fragment thereof, whereas the other vector contains DNA which codes for a co-modulator or a fragment thereof, are exposed to the substance; and

(b) the transcription activity which the nuclear receptor or its fragment induces in the presence of the co-modulator or its fragment and/or the effect of the substance on the interaction between the receptor or its fragment and the co-modulator or its fragment is measured by the protein-protein interaction or the protein-protein-DNA interaction.

The surprising discovery was made that the method according to the invention makes it possible to determine whether or not substances which can be of interest from, for example, an environmental or a pharmacological standpoint exert a hormonal effect, especially an androgenic or anti-androgenic effect, in a reliable, sensitive, simple, rapid, and low-cost manner.

In the process according to the invention, cells which have been transformed with a vector are used. The vectors contain DNA which codes for a nuclear receptor protein or a fragment thereof.

The superfamily of nuclear receptors (NRs), to which more than 50 different proteins belong, is a group of related transcription factors, which control the transcription of the individual target gene in reaction to specific ligands, e.g., hormones. The family can be divided into several subfamilies on the basis of certain characteristics such as dimerization status, type of ligand, and structure of the DNA reaction element (Beato et al., Human Reproduct. Update, Vol.6, pp. 225-236, 2000). A characteristic feature of the NRs is the similarity of the structure of their functional domains (with the designations A-F), consisting of a highly variable, only weakly preserved N-terminal region with an autonomous constitutive activation function (AF-1); a strongly preserved DNA binding domain (DBD), which is responsible for the detection of specific DNA reaction elements and consists of two Zinkfinger motifs; a variable hinge domain; and a preserved, multi-functional C-terminal ligand binding domain (LBD) with a dimerization-dependent and ligand-dependent transactivation function (AF-2). Following after this is the region the farthest away from the C-terminal, the function of which is not known and which is absent in receptors such as PR (progesterone receptor), PPAR (peroxisome proliferator-activated receptor), and RXR (retinoid-X receptor) (Mangelsdorf & Evans, Cell, Vol. 83, pp. 841-850, 1995; Robyr et al., Mol. Endocrinol., Vol. 14, pp. 329-347, 2000). For some of the NRs (e.g., the androgen receptor (AR)), it has been found that the N-terminal region is able to interact with the C-terminal region (Brinkmann et al., J. Steroid Biochem. and Mol. Biol., Vol 69, pp. 307-313, 1999). Steroid hormonal receptors such as estrogen receptor (ER), progesterone receptor (PR), glucocorticoid receptor (GR), mineralocorticoid receptor (MR), and androgen receptor (AR) bind steroidal ligands derived from pregnenolone such as the progestins, the estrogens, the glucocorticoids, the mineralocorticoids, and the androgens. The binding of the ligand activates the receptor and controls the expression of the corresponding target genes.

As explained above, the cells used in step (a) of the method according to the invention contain a vector with a DNA which codes for a co-modulator or a fragment thereof.

The co-modulators are a class of proteins which serve as bridge molecules between the transcription initiation complex and the NRs during the activation (co-activators) or repression (co-repressors) of gene transcription (McKenna et al., Endocr. Rev., Vol. 20, pp. 321-347, 1999). A co-activator must be able to intensify the receptor function and interact directly with the activation domain of NRs in the presence of an agonist. It must also interact with the basic transcription apparatus, but, finally, it may not itself intensify the basic transcription apparatus. Most co-modulators interact with the help of one or more LXXLL motifs (NR boxes) with the AF-2 domain of NRs, but several co-modulators have also been described which interact with other NR regions (Ding et al., Mol. Endocrinol., Vol 12, pp. 302-313, 1998). In addition, many co-modulators have been identified which interact in a similar manner with several different NRs, which suggests that it would be useful to determine the degree of specificity of each co-modulator.

In a preferred embodiment of the method according to the invention, the co-modulator designated ARAP11 or the fragment of ARAP11 containing the amino acids 813-1390 is used. SEQ ID No. 1 and SEQ ID No. 2 show, respectively, the cDNA sequence of the co-modulator ARAP11 and the amino acid sequence of this co-modulator containing the 1390 amino acids. When these proteins are used, it is possible to implement the method according to the invention in an especially reliable, sensitive, simple, low-cost, and rapid manner. In addition, the ARAP11 fragments, especially the fragment of ARAP11 containing amino acids 813-1390, offer the advantage that they are easier to manage and are clonable while still having the functional properties of ARAP11.

ARAP11 is a co-activator for the human androgen receptor and other nuclear receptors; it increases the interaction between an androgen and the receptor. A portion of the sequence of ARAP11 has already been described as Pro2000 in the gene bank XM 005253, but no function is indicated there for it. In comparison with the sequence already known from the gene bank, it has now been established that the amino acid sequence of ARAP11 is larger than the known sequence: it has additional amino acids in the N-terminal region. In addition, it has also been possible to establish that there is an interaction between nuclear receptors, especially AR, and ARAP11, as well as an intensification of AR-mediated transactivation. ARAP11 is a protein which functions as a co-mediator, in that it intensifies or represses the transcription effect after steroids have become bound to the nuclear receptor, and it also promotes the binding and activation of the nuclear receptor to molecules to which no hormonal action was ascribed in the past.

The protein ARAP11 represents a co-activator for the androgen receptor and other nuclear receptors such as estrogen receptor α, estrogen receptor β, progesterone receptor A, progesterone receptor B, glucocorticoid receptor, mineralocorticoid receptor, thyroid hormone receptor, vitamin D receptor, peroxisome proliferator-activated receptor, retinoic acid receptor, retinoid-X receptor, and orphan receptors; in the method according to the invention, these are the preferred receptors, because with them the above-indicated advantages of the method according to the invention can be achieved in an especially favorable manner.

In the process according to the invention, it is also possible to use vectors which code for fragments of the above proteins. “Fragments” in conjunction with the above proteins are understood to be those which have one amino acid or several amino acids less than the full-length proteins but which still have the functional properties of a nuclear receptor or of a co-modulator.

As already explained above, cells which are transfected with two vectors which contain DNA coding for special proteins are used in step (a) of the method according to the invention. These cells are therefore able to express these two different proteins.

The cells are preferably established cell lines and/or eukaryotic cells, especially prostate cells, nerve cells, glial cells, fibroblasts, blood cells, osteoblasts, osteoclasts, hepatocytes, epithelial cells, or muscle cells. By the use of established cell lines, the process according to the invention can be implemented in an especially low-cased and rapid manner. When eukaryotic cells are used, especially the eukaryotic cells listed above, the method according to the invention makes it possible to obtain especially informative results in an advantageous manner.

In a preferred embodiment of the process according to the invention, eukaryotic expression vectors are used such as pCMX or pSG5. When these vectors are used, especially when they are used in conjunction with the above established cell lines and/or eukaryotic cells, the process according to the invention can be carried out especially favorably and quickly, and especially informative results are obtained.

The expert is familiar with methods and the materials required for inserting the DNA coding for the above proteins into a vector, for introducing this vector into the cells, and for cultivating the cells thus obtained under suitable culture conditions so that they can express these proteins.

According to step (b) of the invention, the transcription activity which the nuclear receptor or its fragment induces in the presence of the co-modulator or its fragment is measured. This can be done, for example, by the detection of a reporter gene.

Reporter genes are genes or gene fragments which are coupled with other genes or regulatory sequences in such a way as to make the activity of these sequences detectable. Reporter genes generate gene products which are extremely easy to detect with a photometer as a result of color reactions, for example. Frequently used reporter genes are the gene for β-galactosidase, the gene for alkaline phosphatase, the gene for chloramphenicol acetyl transferase, the gene for catechol dioxygenase, the gene for “green fluorescent protein”, and various luciferase genes, which can cause the cells to produce light.

Such reporter genes can also be introduced into the cells by vectors, especially eukaryotic expression vectors. An example of a vector which contains a reporter gene-coding DNA is the vector MMTV-luciferase, which is used to measure the androgenic effect of substances.

Substances with a hormonal effect, especially with a androgenic/antiandrogenic effect, can then be recognized by the elevated or reduced activity of the reporter gene.

The influence of the test substance on the interaction between the receptor or its fragment and the co-modulator or its fragment can also be measured by determining the protein-protein interaction, e.g., by the use of yeast two hybrid systems, by immunoprecipitation, by GST pull-down assays, by FRET analysis, and by ABCD assays. It can also be measured by determining the protein-protein-DNA interaction by means of gel retardation assays.

It has also been found that ARAP11 can be used very effectively as an indicator of androgen-caused disorders, some of which do not occur until mature years. Relevant androgen-caused disorders such as prostate cancer, erectile dysfunction, infertility, baldness, acne, and hypogonadism and androgen resistance syndromes such as testicular feminization are based on defects in the co-modulation mechanism between AR and ARAP11. A possibility in patients with these types of disorders thus consists in measuring the relative concentrations of AR and ARAP11. This measurement can be made favorably outside the body in body fluids, body cells, or body tissue. This is possible through the use of quantitative methods for measuring the relative quantity of the two molecules in the patient in question, in which methods, for example, antibodies against both AR and against ARAP 11 or nucleic acid probes against their mRNA can be used. There are several methods for measuring these comaprative values, which are known to the expert. The expert also knows suitable materials and devices such as radioimmunoassay, the ELISA test, immunostaining, RT-PCR, Western Blot, Northern Blot, DNA microarrays, and protein microarrays. With the help of ARAP11-cDNA, it is also possible to construct probes in the conventional manner for a PCR assay, by means of which, in certain patients, mutations of the normal DNA sequence can be detected or transcripts for the Northern Blot Assay or a DNA for in-situ hybridization assays can be produced.

The measured ratio of AR to ARAP11 can be greater or less than that present in healthy persons. The normal value of a healthy person can be easily determined by measuring the ratio of AR to ARAP11 in a large number of healthy test subjects. By comparison of the normal value with the ratio of AR to ARAP11 found in the patients to be studied, it can be established whether the value for the determined ratio is greater or less than the normal value.

The concentration of ARAP11 and/or of AR in tissues can vary. For example, it is possible for the concentration of ARAP11 to be very high in the testicles but lower in the liver, heart, thymus, and prostate. It is therefore necessary to take the differing tissue concentrations into consideration when making an evaluation; that is, the test value and the normal value should originate from the same tissue.

Another way in which defects in the co-modulation mechanism between AR and ARAP11 can be determined is to measure only the concentration of ARAP11, it being assumed here that the AR concentration is at least approximately constant. If a lower than normal ARAP11 concentration has been measured, this means that the ratio of AR to ARAP11 has shifted, which serves in turn as an indication of a defect in the co-modulation mechanism.

It is also possible to use an ARAP11-specific probe to determine changes in the expression of ARAP11 and thus changes in the ratio to AR. Such changes can be causally involved in various diseases or occur as a consequence of such diseases.

These types of measurements of the AR/ARAP11 ratio or of ARAP11 are based on the surprising insight, which is based on the discovery and characterization of ARAP11, that an androgen resistance syndrome, for example, can be traced back to a disturbance in the equilibrium between AR and ARAP11 prevalence in the target cells. Too much ARAP11 could lead to a hypersensitivity of the AR system, so that it reacts to molecules which normally do not have any androgenic effect. Conversely, the absence or a malfunction of ARAP11 leads to androgen resistance on all levels. The detection of too much ARAP11 in a patient would suggest the need for down-regulation agents such as antisense or similar medications to reduce the ARAP11 titer in the patient in question under clinical conditions. The same goal can be achieved by molecules which are able to inhibit the interaction between AR and ARAP11. If a patient has too little ARAP11, he can be supplied with ARAP11-cDNA, ARAP11-protein, or ARAP11-DNA via various mechanisms known in and of themselves to increase the titer of active ARAP11. It is also possible to elevate the concentration or the activity of ARAP11 by low-molecular drugs or by stimulation of natural synthesis by means of specific ARAP11-promoter proteins.

As can be seen from the discussion of the method according to the invention presented above, the protein ARAP11 is highly suitable for implementing the method. Another object of the present invention is therefore the ARAP11 with the following amino acid sequence:

```
Met Val Val Leu Arg Ser Ser Leu Glu Leu His Asn His Ser Ala Ala
    1 5 10 15
Ser Ala Thr Gly Ser Leu Asp Leu Ser Ser Asp Phe Leu Ser Leu Glu
                          20 25 30
His Ile Gly Arg Arg Arg Leu Arg Ser Ala Gly Ala Ala Gln Lys Lys
                35 40 45
Pro Ala Ala Thr Thr Ala Lys Ala Gly Asp Gly Ser Ser Val Lys Glu
          50 55 60
Val Glu Thr Tyr His Arg Thr Arg Ala Leu Arg Ser Leu Arg Lys Asp
  65 70 75 80
Ala Gln Asn Ser Ser Asp Ser Ser Phe Glu Lys Asn Val Glu Ile Thr
                                85 90 95
Glu Gln Leu Ala Asn Gly Arg His Phe Thr Arg Gln Leu Ala Arg Gln
                        100 105 110
Gln Ala Asp Lys Lys Lys Glu Glu His Arg Glu Asp Lys Val Ile Pro
                115 120 125
Val Thr Arg Ser Leu Arg Ala Arg Asn Ile Val Gin Ser Thr Glu His
        130 135 140
Leu His Glu Asp Asn Gly Asp Val Glu Val Arg Arg Ser Cys Arg Ile
145 150 155 160
Arg Ser Arg Tyr Ser Gly Val Asn Gln Ser Met Leu Phe Asp Lys Leu
                                165 170 175
Ile Thr Asn Thr Ala Glu Ala Val Leu Gln Lys Met Asp Asp Met Lys
                        180 185 190
Lys Met Arg Arg Gln Arg Met Arg Glu Leu Glu Asp Leu Gly Val Phe
                195 200 205
Asn Glu Thr Glu Glu Ser Asn Leu Asn Met Tyr Thr Arg Gly Lys Gln
        210 215 220
Lys Asp Ile Gln Arg Thr Asp Glu Glu Thr Thr Asp Asn Gln Glu Gly
225 230 235 240
Ser Val Glu Ser Ser Glu Glu Gly Glu Asp Gln Glu His Glu Asp Asp
                                245 250 255
Gly Glu Asp Glu Asp Asp Glu Asp Asp Asp Asp Asp Asp Asp Asp Asp
                        260 265 270
Asp Asp Asp Asp Asp Glu Asp Asp Glu Asp Glu Glu Asp Gly Glu Glu
                275 280 285
Gln Asn Gln Lys Arg Tyr Tyr Leu Arg Gln Arg Lys Ala Thr Val Tyr
        290 295 300
Tyr Gln Ala Pro Leu Glu Lys Pro Arg His Gln Arg Lys Pro Asn Ile
305 310 315 320
Phe Tyr Ser Gly Pro Ala Ser Pro Ala Arg Pro Arg Tyr Arg Leu Ser
                                325 330 335
Ser Ala Gly Pro Arg Ser Pro Tyr Cys Lys Arg Met Asn Arg Arg Arg
                        340 345 350
His Ala Ile His Ser Ser Asp Ser Thr Ser Ser Ser Ser Ser Glu Asp
                355 360 365
Glu Gln His Phe Glu Arg Arg Arg Lys Arg Ser Arg Asn Arg Ala Ile
        370 375 380
Asn Arg Cys Leu Pro Leu Asn Phe Arg Lys Asp Gln Leu Lys Gly Ile
385 390 395 400
Tyr Lys Asp Arg Met Lys Ile Gly Ala Ser Leu Ala Asp Val Asp Pro
                                405 410 415
Met Gln Leu Asp Ser Ser Val Arg Phe Asp Ser Val Gly Gly Leu Ser
                        420 425 430
Asn His Ile Ala Ala Leu Lys Glu Met Val Val Phe Pro Leu Leu Tyr
                435 440 445
Pro Glu Val Phe Glu Lys Phe Lys Ile Gln Pro Pro Arg Gly Cys Leu
        450 455 460
Phe Tyr Gly Pro Pro Gly Thr Gly Lys Thr Leu Val Ala Arg Ala Leu
465 470 475 480
Ala Asn Glu Cys Ser Gln Gly Asp Lys Arg Val Ala Phe Phe Met Arg
                                485 490 495
Lys Gly Ala Asp Cys Leu Ser Lys Trp Val Gly Glu Ser Glu Arg Gln
                        500 505 510
Len Arg Leu Leu Phe Asp Gln Ala Tyr Gln Met Arg Pro Ser Ile Ile
                515 520 525
Phe Phe Asp Glu Ile Asp Gly Leu Ala Pro Val Arg Ser Ser Arg Gln
        530 535 540
Asp Gln Ile His Ser Ser Ile Val Ser Thr Leu Leu Ala Leu Met Asp
545 550 555 560
Gly Leu Asp Ser Arg Gly Glu Ile Val Val Ile Gly Ala Thr Asn Arg
                                565 570 575
Leu Asp Ser Ile Asp Pro Ala Leu Arg Arg Pro Gly Arg Phe Asp Arg
                        580 585 590
Glu Phe Leu Phe Ser Leu Pro Asp Lys Glu Ala Arg Lys Glu Ile Leu
                595 600 605
Lys Ile His Thr Arg Asp Trp Asn Pro Lys Pro Leu Asp Thr Phe Leu
        610 615 620
Glu Glu Leu Ala Glu Asn Cys Val Gly Tyr Cys Gly Ala Asp Ile Lys
625 630 635 640
Ser Ile Cys Ala Glu Ala Ala Leu Cys Ala Leu Arg Arg Arg Tyr Pro
                                645 650 655
Gln Ile Tyr Thr Thr Ser Glu Lys Leu Gln Leu Asp Leu Ser Ser Ile
                        660 665 670
Asn Ile Ser Ala Lys Asp Phe Glu Val Ala Met Gln Lys Met Ile Pro
                675 680 685
Ala Ser Gln Arg Ala Val Thr Ser Pro Gly Gln Ala Leu Ser Thr Val
        690 695 700
Val Lys Pro Leu Leu Gln Asn Thr Val Asp Lys Ile Leu Glu Ala Leu
705 710 715 720
Gln Arg Val Phe Pro His Ala Glu Phe Arg Thr Asn Lys Thr Leu Asp
                                725 730 735
Ser Asp Ile Ser Cys Pro Leu Leu Glu Ser Asp Leu Ala Tyr Ser Asp
                        740 745 750
Asp Asp Val Pro Ser Val Tyr Glu Asn Gly Leu Ser Gln Lys Ser Ser
                755 760 765
His Lys Ala Lys Asp Asn Phe Asn Phe Leu His Leu Asn Arg Asn Ala
        770 775 780
Cys Tyr Gln Pro Met Ser Phe Arg Pro Arg Ile Leu Ile Val Gly Glu
785 790 795 800
Pro Gly Phe Gly Gln Gly Ser His Leu Ala Pro Ala Val Ile His Ala
                                805 810 815
Leu Glu Lys Phe Thr Val Tyr Thr Leu Asp Ile Pro Val Leu Phe Gly
                        820 825 830
Val Ser Thr Thr Ser Pro Glu Glu Thr Cys Ala Gln Val Ile Arg Glu
                835 840 845
Ala Lys Arg Thr Ala Pro Ser Ile Val Tyr Val Pro His Ile His Val
        850 855 860
Trp Trp Glu Ile Val Gly Pro Thr Leu Lys Ala Thr Phe Thr Thr Leu
865 870 875 880
Leu Gln Asn Ile Pro Ser Phe Ala Pro Val Leu Leu Leu Ala Thr Ser
                                885 890 895
Asp Lys Pro His Ser Ala Leu Pro Glu Glu Val Gln Glu Leu Phe Ile
                        900 905 910
Arg Asp Tyr Gly Glu Ile Phe Asn Val Gln Leu Pro Asp Lys Glu Glu
                915 920 925
Arg Thr Lys Phe Phe Glu Asp Leu Ile Leu Lys Gln Ala Ala Lys Pro
        930 935 940
Pro Ile Ser Lys Lys Lys Ala Val Leu Gln Ala Leu Glu Val Leu Pro
945 950 955 960
Val Ala Pro Pro Pro Glu Pro Arg Ser Leu Thr Ala Glu Glu Val Lys
                                965 970 975
Arg Leu Glu Glu Gln Glu Glu Asp Thr Phe Arg Glu Leu Arg Ile Phe
                        980 985 990
Leu Arg Asn Val Thr His Arg Leu Ala Ile Asp Lys Arg Phe Arg Val
                995 1000 1005
Phe Thr Lys Pro Val Asp Pro Asp Glu Val Pro Asp Tyr Val Thr Val
        1010 1015 1020
Ile Lys Gln Pro Met Asp Leu Ser Ser Val Ile Ser Lys Ile Asp Leu
1025 1030 1035 1040
His Lys Tyr Leu Thr Val Lys Asp Tyr Leu Arg Asp Ile Asp Leu Ile
                                1045 1050 1055
Cys Ser Asn Ala Leu Glu Tyr Asn Pro Asp Arg Asp Pro Gly Asp Arg
                      1060 1065 1070
Leu Ile Arg His Arg Ala Cys Ala Leu Arg Asp Thr Ala Tyr Ala Ile
              1075 1080 1085
Ile Lys Glu Glu Leu Asp Glu Asp Phe Glu Gln Leu Cys Glu Glu Ile
      1090 1095 1100
Gln Glu Ser Arg Lys Lys Arg Gly Cys Ser Ser Ser Lys Tyr Ala Pro
1105 1110 1115 1120
Ser Tyr Tyr His Val Met Pro Lys Gln Asn Ser Thr Leu Val Gly Asp
                              1125 1130 1135
Lys Arg Ser Asp Pro Glu Gln Asn Glu Lys Leu Lys Thr Pro Ser Thr
                      1140 1145 1150
Pro Val Ala Cys Ser Thr Pro Ala Gln Leu Lys Arg Lys Ile Arg Lys
              1155 1160 1165
Lys Ser Asn Trp Tyr Leu Gly Thr Ile Lys Lys Arg Arg Lys Ile Ser
      1170 1175 1180
Gln Ala Lys Asp Asp Ser Gln Asn Ala Ile Asp His Lys Ile Glu Ser
1185 1190 1195 1200
Asp Thr Glu Glu Thr Gln Asp Thr Ser Val Asp His Asn Glu Thr Gly
                              1205 1210 1215
Asn Thr Gly Glu Ser Ser Val Glu Glu Asn Glu Lys Gln Gln Asn Ala
                      1220 1225 1230
Ser Glu Ser Lys Leu Glu Leu Arg Asn Asn Ser Asn Thr Cys Asn Ile
              1235 1240 1245
Glu Asn Glu Leu Glu Asp Ser Arg Lys Thr Thr Ala Cys Thr Glu Leu
      1250 1255 1260
Arg Asp Lys Ile Ala Cys Asn Gly Asp Ala Ser Ser Ser Gln Ile Ile
1265 1270 1275 1280
His Ile Ser Asp Glu Asn Glu Gly Lys Glu Met Cys Val Leu Arg Met
                              1285 1290 1295
Thr Arg Ala Arg Arg Ser Gln Val Glu Gln Gln Gln Leu Ile Thr Val
                      1300 1305 1310
Glu Lys Ala Leu Ala Ile Leu Ser Gln Pro Thr Pro Ser Leu Val Val
              1315 1320 1325
Asp His Glu Arg Leu Lys Asn Leu Leu Lys Thr Val Val Lys Lys Ser
      1330 1335 1340
Gln Asn Tyr Asn Ile Phe Gln Leu Glu Asn Leu Tyr Ala Val Ile Ser
1345 1350 1355 1360
Gln Cys Ile Tyr Arg His Arg Lys Asp His Asp Lys Thr Ser Leu Ile
                              1365 1370 1375
Gln Lys Met Glu Gln Glu Val Glu Asn Phe Ser Cys Ser Arg
                      1380 1385 1390
```

or the ARAP11 fragment with amino acids 813-1,390 of this protein.

The object of the present invention is also a DNA coding for ARAP11 or its fragment, especially the fragment with amino acids 813-1,390, and a DNA which hybridizes with them. The term “hybridizing DNA” indicates a DNA which hybridizes with the coding DNA under standard conditions, especially at 20° C. below the melting point of the DNA.

The invention is explained in greater detail below with reference to the following figures, where:

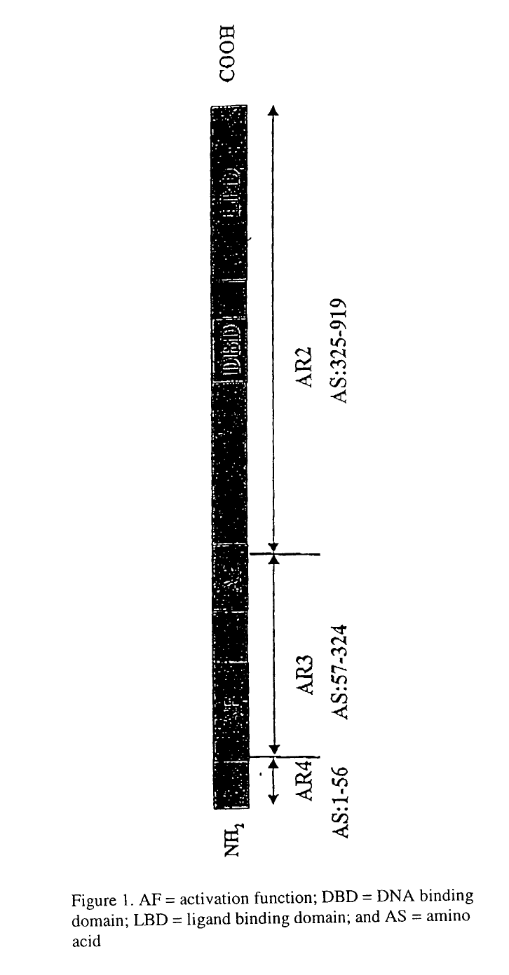

FIG. 1 is a schematic diagram of the androgen receptor with identification of the androgen receptor domain (AR2) extending from amino acid 325 to amino acid 919, this being the domain which is able to interact with ARAP11 in the absence of androgen;

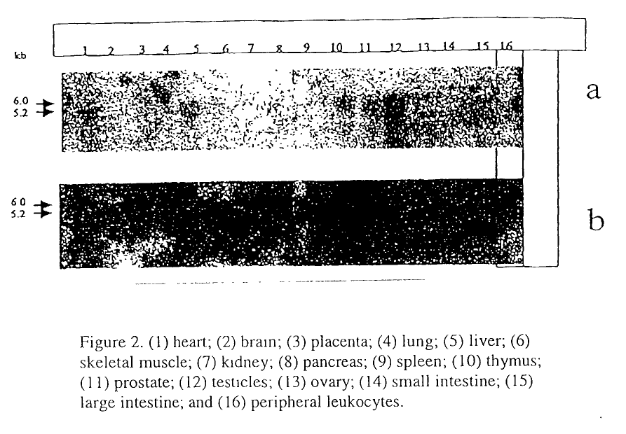

FIG. 2 shows the tissue distribution of ARAP11;

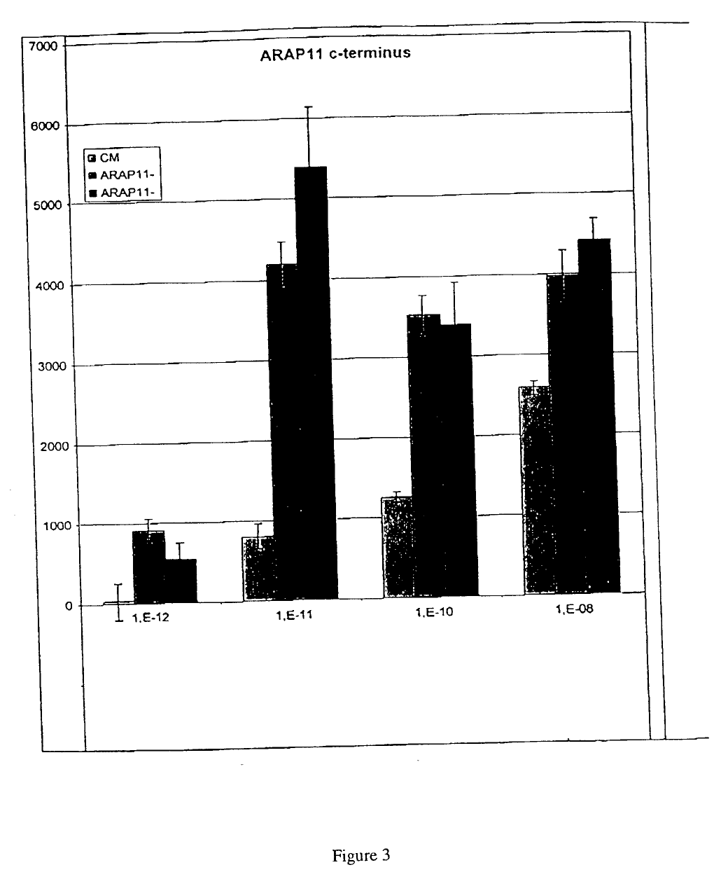

FIG. 3 shows the co-activation of the androgen receptor signal in SH-SY5Y cells; and

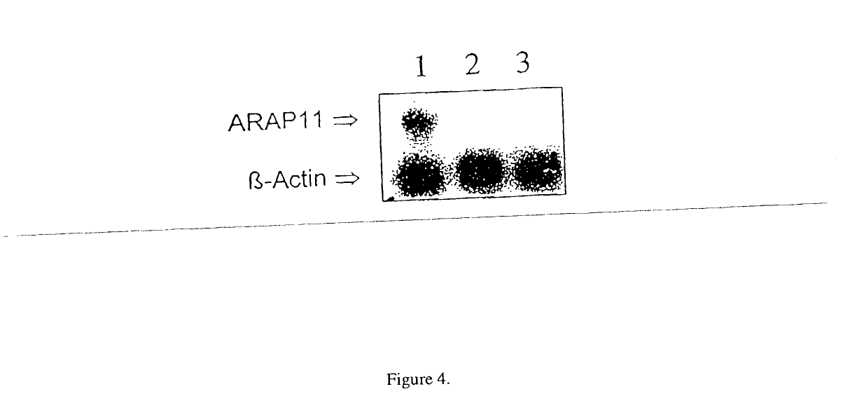

FIG. 4 shows the expression of ARAP11 and β-actin in the testicles of rats.

The following examples illustrate the invention in greater detail without limiting it.

### EXAMPLE 1

### Co-Activation of the Androgen Receptor Signal by ARAP11

With the use of a cDNA library from fetal brain (Clontech MATCHMAKER) and of a human AR fragment which codes for amino acids 325-919 as a probe (FIG. 1), a screening process was carried out by means of a conventional two-hybrid yeast system in the absence of androgen. In agreement with the instructions of the manufacturer (Clontech), the number of screened clones was 6×10⁷. The number of independent clones according to the manufacturer was 3.5×10⁸. From these, 350 positive clones were selected and tested by a β-galactosidase assay; 240 were confirmed as being lacZ-positive. The inserts of these clones were amplified by PCR. At least 17 different clones were identified by restriction fragment analyses and sequencing. One of these was a clone with an insert comprising 1,169 bp (3,243 bp-4,412 bp), which codes for a part of the ORF (Open Reading Frame). This sequence also contains almost the entire part of the ORF already described in Pro2000 (Gene Bank Access No. XM005253).

By means of a conventional PCR method, the coding ARAP11-cDNA which codes for a protein (SEQ ID No. 2) consisting of 1390 amino acids and extending considerably beyond the previously know Pro 2000 sequence, which describes a protein with 362 amino acids, was cloned in its full length. Together with the 5′ and 3′ nontranslated regions, the sequence described here has a length of 4,412 bp (SEQ ID No. 1).

FIG. 2 shows the tissue distribution of ARAP11, which was studied by means of Northern Blot analysis in the standard manner. Poly-A+-RNA (2 μg) isolated from various human tissues was separated by a formaldehyde-containing agarose gel, blotted onto a Nylon membrane, and hybridized with a labeled ARAP11-cDNA fragment. For the experiment illustrated in FIGS. 2a and 2b, a fragment of 3,111-4,217 bp of the cDNA sequence of ARAP11 was used; for the experiment illustrated in FIG. 2c, a fragment of 2,065-2,476 bp was used. After washing, the membrane was laid on a piece of film and developed after exposure to light for either 24 hours (FIGS. 2a and 2c) or 8 days (FIG. 2b). As can be seen from FIG. 2, very strong expression of ARAP11 was detected in the testicles, whereas weaker expression was found in the liver, in the heart, in the thymus, and in the prostate. Two transcripts (6.0 kb and 5.2 kb) were discovered.

When the probe consisting of the fragment with 2,065-2,476 bp of the cDNA sequence of ARAP11, containing 411 bp, was used, two transcripts of equal size were again found in the testicles. It can therefore be assumed that the transcripts which were found are identical to the transcripts which were detected in 2a and 2b with the probe of 3,111-4,217. This is evidence that the sequence of Pro 2000 filed in the gene bank under XM005253 is incomplete and is 2,480 bp longer in the 5′ region.

The ARAP11-cDNA which codes for the ARAP11 fragment of amino acids 813-1,390, obtained by PCR, was cloned in the standard manner into the vector CMX and transfected with pSG5-AR and MMTV-luciferase in SH-SY5Y cells, also in the standard manner.

As can be seen from FIG. 3, the transient transfection of ARAP11-cDNA in SH-SYT5Y cells led to a strong co-activation of the AR signal activity, especially at low androgen concentrations of 10⁻¹²-10⁻¹⁰ M. For this purpose, in a cell culture tray with wells, 3×10⁵ cells per well were transfected with 1 μg of co-activator (ARAP11-3 or ARAP11-1, coding in each case for amino acids 813-1,390 of ARAP11) in CMX or with 1 μg of CMX as control plasmid, with 1.5 μg of MMTV-luciferase plasmid, and with 0.75 μg of pSG5AR plasmid. After 24 hours, the cells were treated with dihydroxytestosterone (DHT) as the androgen in the indicated concentrations. The transfected cells were harvested after another 24 hours, and the activity of the reporter gene luciferase was measured. In addition, the total quantity of cell protein was determined for the sake of normalization. One experiment and four measurements were conducted per transfection batch and substance concentration. The error range is given as the SD. The values of the corresponding controls without DHT were subtracted from all signals. The activity is expressed in relative units.

### EXAMPLE 2

### Determination of ARAP11 in the Testicles of Rats

FIG. 4 shows the expression of ARAP 11 and β-actin in the testicles of rats. Poly-A+RNAs (4 μg) were isolated from the tissues of rat testicles, separated with a formaldehyde-containing agarose gel, transferred to a Nylon membrane, and hybridized either with a labeled ARAP11-cDNA fragment (2,226-4,228 bp) or with a labeled β-actin CDNA (rat). After washing, the membrane was laid on a piece of film, exposed to light for 5 days, and developed. A RNA transcript (6.0 kb) could be detected in the testicular tissue of the rats. The isolated RNA of 3-week-old animals is plotted in column 1, that of 6-week-old animals in column 2, and that of 2-year-old animals in column 3. It is easy to see that the expression of the ARAP11 gene is clearly age-dependent, whereas the expression of the β-actin gene shows no change. Six weeks after birth, the expression of ARAP11 is considerably reduced (by more than 50%), and in old animals (2 years) only a very low level of expression of the ARAP11 gene can still be detected. Similar behavior in terms of changes in the gene expression of the co-modulator ARAP11 can be expected in certain disease pictures.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents.

All references cited herein, including patents, patent applications, papers, text books, and the like, and the references cited therein, to the extent that they are not already, are hereby incorporated herein by reference in their entirety.

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method for testing the hormonal effect, especially the androgenic or antiandrogenic effect, of substances, in which
(a) cells which have been transfected with two vectors, one of these vectors containing DNA which codes for a nuclear receptor protein or a fragment thereof, the other vector containing DNA which codes for a co-modulator or a fragment thereof, are exposed to the substance; and
(b) the transcription activity which the nuclear receptor or its fragment induces in the presence of the co-modulator or its fragment and/or the effect of the substance on the interaction between the receptor or its fragment and the co-modulator or its fragment is measured by the protein-protein interaction or protein-protein-DNA interaction.

2. The method according to claim 1, where the co-modulator is ARAP11, which comprises the following amino acid sequence:
```
Met Val Val Leu Arg Ser Ser Leu Glu Leu His Asn His Ser Ala Ala
    15 10 15
Ser Ala Thr Gly Ser Leu Asp Leu Ser Ser Asp Phe Leu Ser Leu Glu
                        20 25 30
His Ile Gly Arg Arg Arg Leu Arg Ser Ala Gly Ala Ala Gln Lys Lys
                35 40 45
Pro Ala Ala Thr Thr Ala Lys Ala Gly Asp Gly Ser Ser Val Lys Glu
          50 55 60
Val Glu Thr Tyr His Arg Thr Arg Ala Leu Arg Ser Leu Arg Lys Asp
  65 70 75 80
Ala Gln Asn Ser Ser Asp Ser Ser Phe Glu Lys Asn Val Glu Ile Thr
                                  85 90 95
Glu Gln Leu Ala Asn Gly Arg His Phe Thr Arg Gln Leu Ala Arg Gln
                        100 105 110
Gln Ala Asp Lys Lys Lys Glu Glu His Arg Glu Asp Lys Val Ile Pro
                115 120 125
Val Thr Arg Ser Leu Arg Ala Arg Asn Ile Val Gln Ser Thr Glu His
        130 135 140
Leu His Glu Asp Asn Gly Asp Val Glu Val Arg Arg Ser Cys Arg Ile
145 150 155 160
Arg Ser Arg Tyr Ser Gly Val Asn Gln Ser Met Leu Phe Asp Lys Leu
                                165 170 175
Ile Thr Asn Thr Ala Glu Ala Val Leu Gln Lys Met Asp Asp Met Lys
                        180 185 190
Lys Met Arg Arg Gln Arg Met Arg Glu Leu Gln Asp Leu Gly Val Phe
                195 200 205
Asn Glu Thr Glu Glu Ser Asn Leu Asn Met Tyr Thr Arg Gly Lys Gln
        210 215 220
Lys Asp Ile Gln Arg Thr Asp Glu Glu Thr Thr Asp Asn Gln Glu Gly
225 230 235 240
Ser Val Glu Ser Ser Glu Glu Gly Glu Asp Gln Glu His Glu Asp Asp
                                245 250 255
Gly Glu Asp Glu Asp Asp Glu Asp Asp Asp Asp Asp Asp Asp Asp Asp
                        260 265 270
Asp Asp Asp Asp Asp Glu Asp Asp Glu Asp Glu Glu Asp Gly Glu Glu
                275 280 285
Glu Asn Gln Lys Arg Tyr Tyr Leu Arg Gln Arg Lys Ala Thr Val Tyr
        290 295 300
Tyr Gln Ala Pro Leu Glu Lys Pro Arg His Gln Arg Lys Pro Asn Ile
305 310 315 320
Phe Tyr Ser Gly Pro Ala Ser Pro Ala Arg Pro Arg Tyr Arg Leu Ser
                                325 330 335
Ser Ala Gly Pro Arg Ser Pro Tyr Cys Lys Arg Met Asn Arg Arg Arg
                        340 345 350
His Ala Ile His Ser Ser Asp Ser Thr Ser Ser Ser Ser Ser Glu Asp
                355 360 365
Glu Gln His Phe Glu Arg Arg Arg Lys Arg Ser Arg Asn Arg Ala Ile
        370 375 380
Asn Arg Cys Leu Pro Leu Asn Phe Arg Lys Asp Glu Leu Lys Gly Ile
385 390 395 400
Tyr Lys Asp Arg Met Lys Ile Gly Ala Ser Leu Ala Asp Val Asp Pro
                                405 410 415
Met Gln Leu Asp Ser Ser Val Arg Phe Asp Ser Val Gly Gly Leu Ser
                        420 425 430
Asn His Ile Ala Ala Leu Lys Glu Met Val Val Phe Pro Leu Leu Tyr
                435 440 445
Pro Glu Val Phe Glu Lys Phe Lys Ile Gln Pro Pro Arg Gly Cys Leu
        450 455 460
Phe Tyr Gly Pro Pro Gly Thr Gly Lys Thr Leu Val Ala Arg Ala Leu
465 470 475 480
Ala Asn Glu Cys Ser Gln Gly Asp Lys Arg Val Ala Phe Phe Met Arg
                                485 490 495
Lys Gly Ala Asp Cys Leu Ser Lys Trp Val Gly Glu Ser Glu Arg Gln
                        500 505 510
Leu Arg Leu Leu Phe Asp Gln Ala Tyr Gln Met Arg Pro Ser Ile Ile
                515 520 525
Phe Phe Asp Glu Ile Asp Gly Leu Ala Pro Val Arg Ser Ser Arg Gln
        530 535 540
Asp Gln Ile His Ser Ser Ile Val Ser Thr Leu Leu Ala Leu Met Asp
545 550 555 560
Gly Leu Asp Ser Arg Gly Glu Ile Val Val Ile Gly Ala Thr Asn Arg
                                565 570 575
Leu Asp Ser Ile Asp Pro Ala Leu Arg Arg Pro Gly Arg Phe Asp Arg
                        580 585 590
Glu Phe Leu Phe Ser Leu Pro Asp Lys Glu Ala Arg Lys Glu Ile Leu
                595 600 605
Lys Ile His Thr Arg Asp Trp Asn Pro Lys Pro Leu Asp Thr Phe Leu
        610 615 620
Glu Glu Leu Ala Glu Asn Cys Val Gly Tyr Cys Gly Ala Asp Ile Lys
625 630 635 640
Ser Ile Cys Ala Glu Ala Ala Leu Cys Ala Leu Arg Arg Arg Tyr Pro
                                645 650 655
Gln Ile Tyr Thr Thr Ser Glu Lys Leu Gln Leu Asp Leu Ser Ser Ile
                        660 665 670
Asn Ile Ser Ala Lys Asp Phe Glu Val Ala Met Gln Lys Met Ile Pro
                675 680 685
Ala Ser Gln Arg Ala Val Thr Ser Pro Gly Gln Ala Leu Ser Thr Val
        690 695 700
Val Lys Pro Leu Leu Gln Asn Thr Val Asp Lys Ile Leu Glu Ala Leu
705 710 715 720
Gln Arg Val Phe Pro His Ala Glu Phe Arg Thr Asn Lys Thr Leu Asp
                                725 730 735
Ser Asp Ile Ser Cys Pro Leu Leu Glu Ser Asp Leu Ala Tyr Ser Asp
                        740 745 750
Asp Asp Val Pro Ser Val Tyr Glu Asn Gly Leu Ser Gln Lys Ser Ser
                755 760 765
His Lys Ala Lys Asp Asn Phe Asn Phe Leu His Leu Asn Arg Asn Ala
        770 775 780
Cys Tyr Gln Pro Met Ser Phe Arg Pro Arg Ile Leu Ile Val Gly Glu
785 790 795 800
Pro Gly Phe Gly Gln Gly Ser His Leu Ala Pro Ala Val Ile His Ala
                                805 810 815
Leu Glu Lys Phe Thr Val Tyr Thr Leu Asp Ile Pro Val Leu Phe Gly
                        820 825 830
Val Ser Thr Thr Ser Pro Glu Glu Thr Cys Ala Gln Val Ile Arg Glu
                835 840 845
Ala Lys Arg Thr Ala Pro Ser Ile Val Tyr Val Pro His Ile His Val
        850 855 860
Trp Trp Glu Ile Val Gly Pro Thr Leu Lys Ala Thr Phe Thr Thr Leu
865 870 875 880
Leu Gln Asn Ile Pro Ser Phe Ala Pro Val Leu Leu Leu Ala Thr Ser
                                885 890 895
Asp Lys Pro His Ser Ala Leu Pro Glu Glu Val Gln Glu Leu Phe Ile
                        900 905 910
Arg Asp Tyr Gly Glu Ile Phe Asn Val Gln Leu Pro Asp Lys Glu Glu
                915 920 925
Arg Thr Lys Phe Phe Glu Asp Leu Ile Leu Lys Gln Ala Ala Lys Pro
        930 935 940
Pro Ile Ser Lys Lys Lys Ala Val Leu Gln Ala Leu Glu Val Leu Pro
945 950 955 960
Val Ala Pro Pro Pro Glu Pro Arg Ser Leu Thr Ala Glu Glu Val Lys
                                965 970 975
Arg Leu Glu Glu Gln Glu Glu Asp Thr Phe Arg Glu Leu Arg Ile Phe
                        980 985 990
Leu Arg Asn Val Thr His Arg Leu Ala Ile Asp Lys Arg Phe Arg Val
                995 1000 1005
Phe Thr Lys Pro Val Asp Pro Asp Glu Val Pro Asp Tyr Val Thr Val
        1010 1015 1020
Ile Lys Gln Pro Met Asp Leu Ser Ser Val Ile Ser Lys Ile Asp Leu
1025 1030 1035 1040
His Lys Tyr Leu Thr Val Lys Asp Tyr Leu Arg Asp Ile Asp Leu Ile
                                1045 1050 1055
Cys Ser Asn Ala Leu Glu Tyr Asn Pro Asp Arg Asp Pro Gly Asp Arg
                        1060 1065 1070
Leu Ile Arg His Arg Ala Cys Ala Leu Arg Asp Thr Ala Tyr Ala Ile
                1075 1080 1085
Ile Lys Glu Glu Leu Asp Glu Asp Phe Glu Gln Leu Cys Glu Glu Ile
      1090 1095 1100
Gln Glu Ser Arg Lys Lys Arg Gly Cys Ser Ser Ser Lys Tyr Ala Pro
1105 1110 1115 1120
Ser Tyr Tyr His Val Met Pro Lys Gln Asn Ser Thr Leu Val Gly Asp
                              1125 1130 1135
Lys Arg Ser ASP Pro Glu Gln Asn Glu Lys Leu Lys Thr Pro Ser Thr
                      1140 1145 1150
Pro Val Ala Cys Ser Thr Pro Ala Gln Leu Lys Arg Lys Ile Arg Lys
              1155 1160 1165
Lys Ser Asn Trp Tyr Leu Gly Thr Ile Lys Lys Arg Arg Lys Ile Ser
      1170 1175 1180
Gln Ala Lys Asp Asp Ser Gln Asn Ala Ile Asp His Lys Ile Glu Ser
1185 1190 1195 1200
Asp Thr Glu Glu Thr Gln Asp Thr Ser Val Asp His Asn Glu Thr Gly
                              1205 1210 1215
Asn Thr Gly Glu Ser Ser Val Glu Glu Asn Glu Lys Gln Gln Asn Ala
                      1220 1225 1230
Ser Glu Ser Lys Leu Glu Leu Arg Asn Asn Ser Asn Thr Cys Asn Ile
              1235 1240 1245
Glu Asn Glu Leu Glu Asp Ser Arg Lys Thr Thr Ala Cys Thr Glu Leu
      1250 1255 1260
Arg Asp Lys Ile Ala Cys Asn Gly Asp Ala Ser Ser Ser Gln Ile Ile
1265 1270 1275 1280
His Ile Ser Asp Glu Asn Glu Gly Lys Glu Met Cys Val Leu Arg Met
                              1285 1290 1295
Thr Arg Ala Arg Arg Ser Gln Val Glu Gln Gln Gln Leu Ile Thr Val
                      1300 1305 1310
Glu Lys Ala Leu Ala Ile Leu Ser Gln Pro Thr Pro Ser Leu Val Val
              1315 1320 1325
Asp His Glu Arg Leu Lys Asn Leu Leu Lys Thr Val Val Lys Lys Ser
      1330 1335 1340
Gln Asn Tyr Asn Ile Phe Gln Leu Glu Asn Leu Tyr Ala Val Ile Ser
1345 1350 1355 1360
Gln Cys Ile Tyr Arg His Arg Lys Asp His Asp Lys Thr Ser Leu Ile
                              1365 1370 1375
Gln Lys Met Glu Gln Glu Val Glu Asn Phe Ser Cys Ser Arg
                      1380 1385 1390
```

3. The method according to claim 1, where the fragment of the co-modulator contains the amino acids 813-1390 of ARAP11.

4. The method according to claims 1, 2 or 3, where the nuclear receptor is selected from androgen receptor, estrogen receptor α, estrogen receptor β, progesterone receptor A, progesterone receptor B, glucocorticoid receptor, mineralocorticoid receptor, thyroid hormone receptor, vitamin D receptor, peroxisome proliferator-activated receptor, retinoic acid receptor, retinoid X receptor, and orphan receptors.

5. The method according to claim 4 where the cells are established cell lines and/or eukaryotic cells.

6. The method according to claim 5, where the eukaryotic cells are selected from prostate cells, nerve cells, glial cells, fibroblasts, blood cells, osteoblasts, osteoclasts, hepatocytes, epithelial cells, or muscle cells.

7. The method according to claims 1, 2, or 3 where the vector is a eukaryotic expression vector.

8. A method for determining defects in the co-modulation mechanism between androgen receptors and ARAP11, wherein the concentrations of ARAP11 or a fragment thereof and of androgen receptor and/or a fragment thereof are measured.

9. The method according to claim 8, where the concentration measurement is carried out by radioimmunoassay, an ELISA test, immunostaining, RT-PCR, Western Blot, Northern Blot, DNA microarrays, or protein microarrays.

10. A protein or a fragment thereof with co-modulator properties for the androgen receptor, having the amino acid sequence according to claim 2.

11. The protein according to claim 10, where the fragment contains amino acids 813-1390.

12. A DNA sequence coding for the proteins according to claim 10 or 11 or DNA hybridizing with said DNA sequence.

13. A method for testing the hormonal or anti-hormonal effect of a chemical compound in vitro comprising the steps of:
(a) providing cells which are transfected with two vectors, wherein one of said vectors contains DNA coding for a nuclear receptor protein or a fragment thereof, especially a human nuclear receptor or a fragment thereof, and the other vector contains DNA which codes for a co-modulator or a fragment thereof;
(b) exposing the transformed host cells to the chemical compound; and
(c) measuring the level of transcriptional activity caused by the hormone receptor.

14. The method according to claim 13, where the co-modulator is ARAP11, having the amino acid sequence SEQ ID No. 2.

15. The method according to claim 13, where the fragment of the co-modulator contains the amino acids 813-1390 of ARAP11.

16. The method according to claims 13, 14 or 15, where the nuclear receptor is selected from androgen receptor, estrogen receptor α, estrogen receptor β, progesterone receptor A, progesterone receptor B, glucocorticoid receptor, mineralocorticoid receptor, thyroid hormone receptor, vitamin D receptor, peroxisome proliferator-activated receptor, retinoic acid receptor, retinoid X receptor, and orphan receptors.

17. The method according to claim 16 wherein the cells are established cell lines and/or eukaryotic cells.

18. The method according to claim 17, wherein the eukaryotic cells are selected from the group consisting of prostate cells, nerve cells, glial cells, fibroblasts, blood cells, osteoblasts, osteoclasts, hepatocytes, epithelial cells, or muscle cells.

19. The method according to claims 13, 14, or 15 where the vector is a eukaryotic expression vector.

20. A method for testing the androgenic or antiandrogenic effect of a chemical compound in vitro comprising the steps of:
(a) transforming host cells with a genetic construct effective in that host cell to produce both human androgen receptor protein and ARAP11 protein;
(b) exposing the transformed host cells to the chemical compound; and
(c) measuring the level of transcriptional activity caused by said androgen receptor.

21. The method of claim 20 wherein the host cells are selected from the group consisting of prostate cells, nerve cells, glial cells, fibroblasts, blood cells, osteoblasts, osteoclasts, hepatocytes, epithelial cells, or muscle cells.

22. The method of claim 20 wherein the genetic construct producing the ARAP11 protein has the DNA sequence of SEQ ID NO. 1.

23. The method of claim 20 wherein the genetic construct also includes a reporter gene, the expression of which can be detected and quantified.

24. The method of claim 20 wherein the chemical compound is a pharmaceutical.

25. The method of claim 20 wherein the chemical compound is contained in an environmental sample.

26. The method of claim 23 wherein the reporter gene is selected from the group consisting of: the gene for β-galactosidase, the gene for alkaline phosphatase, the gene for chloramphenicol acetyl transferase, the gene for catechol dioxygenase, the gene for “green fluorescent protein”, and the luciferase genes.
